(19) United States
(12) Patent Application Publication
(10) Pub. No.: US 2015/0050313 A1
(43) Pub. Date: Feb. 19, 2015

(54) NOVEL IMMUNOSTIMULANTS AND SYNTHESIS THEREOF

(71) Applicant: The Regents of the University of California, Oakland, CA (US)

(72) Inventors: Rock J. Mancini, Irvine, CA (US); Janine K. Tom, Irvine, CA (US); Aaron P. Esser-Kahn, Irvine, CA (US)

(21) Appl. No.: 14/458,988

(22) Filed: Aug. 13, 2014

Related U.S. Application Data
(60) Provisional application No. 61/866,726, filed on Aug. 16, 2013.

(51) Int. Cl. C07H 21/04; A61K 31/80; C08G 65/335

(52) U.S. Cl. CPC C07H 21/04; C08G 65/3356; A61K 31/80
USPC 424/193.1; 536/23.1; 435/375

(57) ABSTRACT

Disclosed herein are heteromeric molecules comprising two or more different pathogen associated molecular patterns (PAMPs), wherein the two or more PAMPs are linked together with discrete, linear inter-PAMP spacing. Also disclosed are immunogenic compositions comprising the heteromeric molecules and related methods of stimulating an immune response in vivo in a subject or ex vivo comprising administering the immunogenic composition to cells. Also disclosed is a method of synthesizing an immunostimulant comprising: identifying 2 or more different PAMPS, covalently linking the 2 or more different PAMPS together, varying spacing between the 2 or more different PAMPs, and testing the effect of spacing between the 2 or more different PAMPS to identify an optimal spacing that results in immune stimulation.

### FIELD OF THE INVENTION

The disclosure is broadly applicable to the field of immunology and is particularly useful in cancer immunotherapy or vaccine formulation.

### BACKGROUND OF THE INVENTION

Immunotherapies are a promising technology for interacting with the immune system. Vaccines, the most successful immunotherapies, operate through synergistic stimulation of dendritic cell receptors (Banchereau, J. and Steinman, R. M. 1998 Nature 392: 245-252) using spatio-controlled presentation of pathogen associated molecular patterns (PAMPs) in the form of attenuated whole viruses. The response pathway of many individual receptors (Akira, S. et al. 2001 Nat Immunol 2: 675-680) and some synergistic PAMP combinations result in increased stimulation (Shukla, N. M. et al. 2012 J Med Chem 55: 1106-1116). However, the activity of PAMP-heterodimers with discrete inter-PAMP spacing synthetically controlled by polymeric linkers has never been tested.

Pathogen associated molecular patterns (PAMPs) are the molecular signals that the immune system uses to determine self from non-self, and immunostimulatory synergies between PAMPs are emerging as a key aspect in applications ranging from vaccine formulation to cancer immunotherapy. For example, in attenuated whole virus vaccines, these synergies are the key to the generation of robust immune responses and life-long immunity. (Akira, S. et al. 2004 Nat Rev Immunol 4: 499-511; Takeda, K et al. 2003 Annu Rev Immunol 21:335-376; Banchereau, J. and Steinman, R. M. 1998 Nature 392:245-252; Banchereau, J. et al. 2000 Annual Review of Immunology 18:767-811; Steinman, R. M. 1991 Annual Review of Immunology 9:271-296). Simultaneous stimulation of elpitlum toll-like receptors (TLRs) on the surface of dendritic cells (DCs) or within endosomal compartments directs the level and polarization. of the immune response. Previous attempts to mimic the synergistic stimulation motif of viruses or other pathogens have involved particle or macromolecular delivery systems containing multiple PAMPs. However, few combinations reach the same level of stimulation as live or attenuated vaccines, and the polarization of the immune response is often unknown or uncharacterized.

The molecular basis for the activation of TLRs has also been investigated. In particular x-ray crystallography and fluorescence imaging suggest that synergistic signaling occurs when certain sets of TLRs undergo PAMP-mediated association as hetero or homodimers. This causes oligomerization of myeloid differentiation primary response gene 88 (MYD88) into a multi-protein construct termed the mydozome which can cause up-regulation of NFκB, a general marker of immune-cell activation. We hypothesized that TLR synergies in the activation of the mydozome might be tested or enhanced by creating PAMP heterodimers using a polymeric linker analogous to induced chemical dimerization. This encourages the association of multiple TLRs, further promoting MYD88 oligomerization.

Control of the immune system is required for effective vaccines and immunotherapies. Single immunostimulants alone do not provide the complete set of immune signals required to activate the desired immune responses (immunity, antibody titers, tumor clearance). Present solutions include mixtures of immunostimulants, combination immunostimulants and covalent attachment of antigens to growth factors, such as GMCSF. Simply mixing immunostimulants does generate an increased immune response, however, the magnitude of the synergy of stimulation is limited and it is difficult to predict the polarization of the immune response. Active portions of various immunostimulants have been covalently attached to each other to provide increased immune stimulation. However, the attachment scheme is not generally applicable to all currently known immunostimulant combinations. Therefore, only agonists of a select subset of activation pathways have been combined. Additionally, the molecules comprise combinations of only known active portions of currently known immunostimulants. This has been shown to alter activation and polarization of the immune response. Lastly, the distance between the two stimulants has not been varied, and cannot be easily varied via the current state-of-the-art methods. Although direct attachment of antigen to GMCSF does generate an immune response towards the selected antigen, the magnitude of the response is low.

### SUMMARY OF THE INVENTION

This heteromeric molecules, compositions and methods disclosed herein increase the response of the immune system at lower concentrations than currently available technologies. They can be used to direct the immune system. As such, the heteromeric molecules, compositions and methods disclosed herein will be useful in generating a long-lasting immune response desired in vaccines. Additionally, the heteromeric molecules and compositions will be useful in cancer immunotherapy formulations to activate the immune system in vivo or ex vivo towards various types of tumors or cancers.

Disclosed herein are heteromeric molecules comprising two or more different pathogen associated molecular patterns (PAMPs), wherein the two or more PAMPs are linked together with discrete, linear inter-PAMP spacing.

In some embodiments, the two or more different PAMPs are linked together with a heterotelechelic polymer.

In some embodiments, the linear inter-PAMP spacing is from 10 to 100 Å

In some embodiments, the linear inter-PAMP spacing is from 32.5 to 95.6 Å.

In some embodiments, the two or more different PAMPs comprise lipoteichoic acid and a CpG-DNA fragment.

Also disclosed is an immunogenic composition comprising the heteromeric molecules comprising two or more different pathogen associated molecular patterns (PAMPs), wherein the two or more PAMPs are linked together with discrete, linear inter-PAMP spacing.

Also disclosed is a method of stimulating an immune response in vivo in a subject comprising administering the immunogenic composition to the subject.

In some embodiments, the subject has a tumor or a cancer.

Also disclosed is a method of stimulating an immune response ex vivo comprising administering the immunogenic composition to cells.

Also disclosed is a method of synthesizing an immunostimulant comprising: identifying 2 or more different PAMPS, covalently linking the 2 or more different PAMPS together, varying spacing between the 2 or more different PAMPs, and testing the effect of spacing between the 2 or more different PAMPS to identify an optimal spacing that results in immune stimulation.

### BRIEF DESCRIPTION OF THE DRAWINGS

FIG. 1. Synthesis of PAMP-Heterodimers. a) LTA was conjugated to an α,ω-eterotelechelic PEG based linker via reaction of the NHS-ester end-group on the PEG linker with amines in alanine residues present in the LTA backbone. The LTA-PEG conjugate was then treated with a thiolated CpG DNA to effect conjugation with the maleimide end-group thereby completing functionalization of the PAMP-heterodimer. b) The resulting CpG-LTA PAMP-heterodimers were purified by FPLC (1×TBE, n=6 shown) and characterized via standard methods (UV/Vis, MS, SDS-PAGE).

FIG. 2. Synergistic Immunostimulation. a) RAW-Blue cells were stimulated by addition of CpG, LTA, a mixture of the two PAMPs, or the LTA-PEG6-CpG PAMP heterodimer. After 15 h, the colorimetric QUANTI-BLUE™ assay (Invivogen, CA) was used to quantify SEAP as a measure of NFκB activation. b) In a similar experiment RAW-Blue cells were stimulated with the LTA-PEG6-CpG and LTA-PEG24-CpG heterodimers or LPS. The QUANTI-BLUE™ assay was again used to determine NFκB activation (results are normalized to LPS at 10 μg/mL).

FIG. 3. Immune Response and Polarization. a) BMDCs were stimulated by addition of CpG, LTA, a mixture of the two PAMPs, or the LTA-PEG6-CpG heterodimer at a concentration of 100 ng/mL with respect to CpG. After 18 h, cell surface proteins involved with T-cell expansion were observed via immunohistochemical staining. All activation signals were normalized to the resting state except for CD 86, which was normalized to the isotype control.

FIG. 4. Inter-PAMP Synergies. Native inter-PAMP spacings are present in pathogens such as viral capsids, and previous work has probed the synergistic immunostimulatory effects of PAMP combinations such as LTA and CpG in unconjugated mixtures. We have conjugated LTA and CpG as a CpG-LTA PAMP-heterodimer mimicking the native inter-PAMP spacing.

FIG. 5. From experiment to understanding: knowing how the immune system works has come a long way.

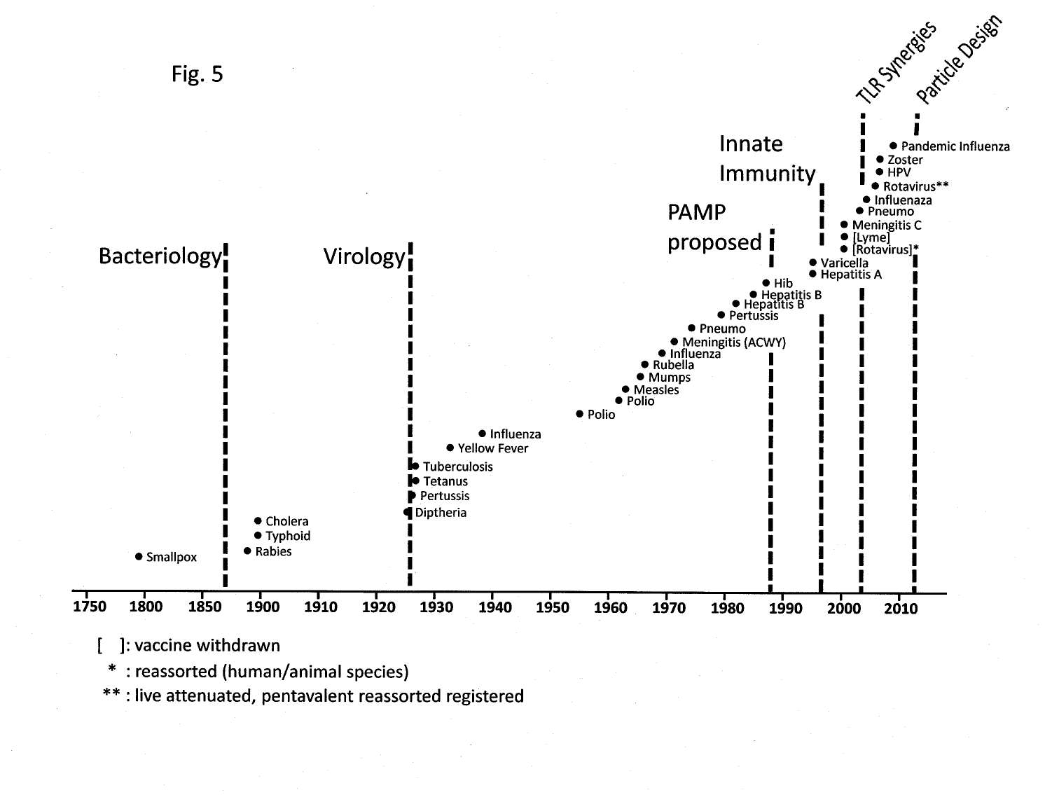

FIG. 6. Development of the Yellow Fever Virus Vaccine-Life Long Immunity.

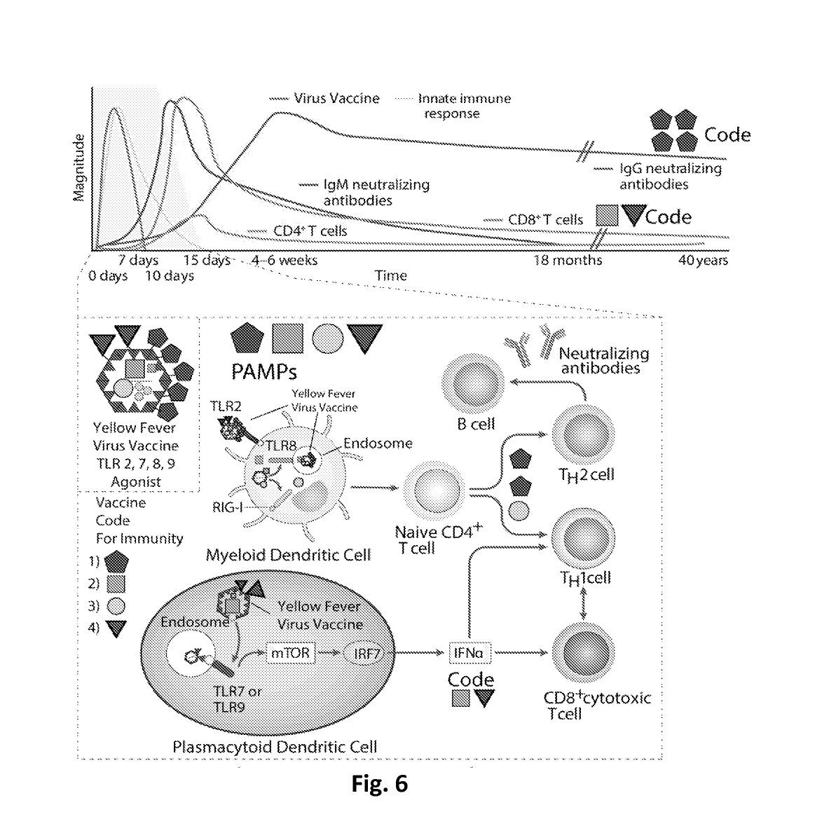

FIG. 7. Different organisms have different PAMPs that trigger multiple TLRs.

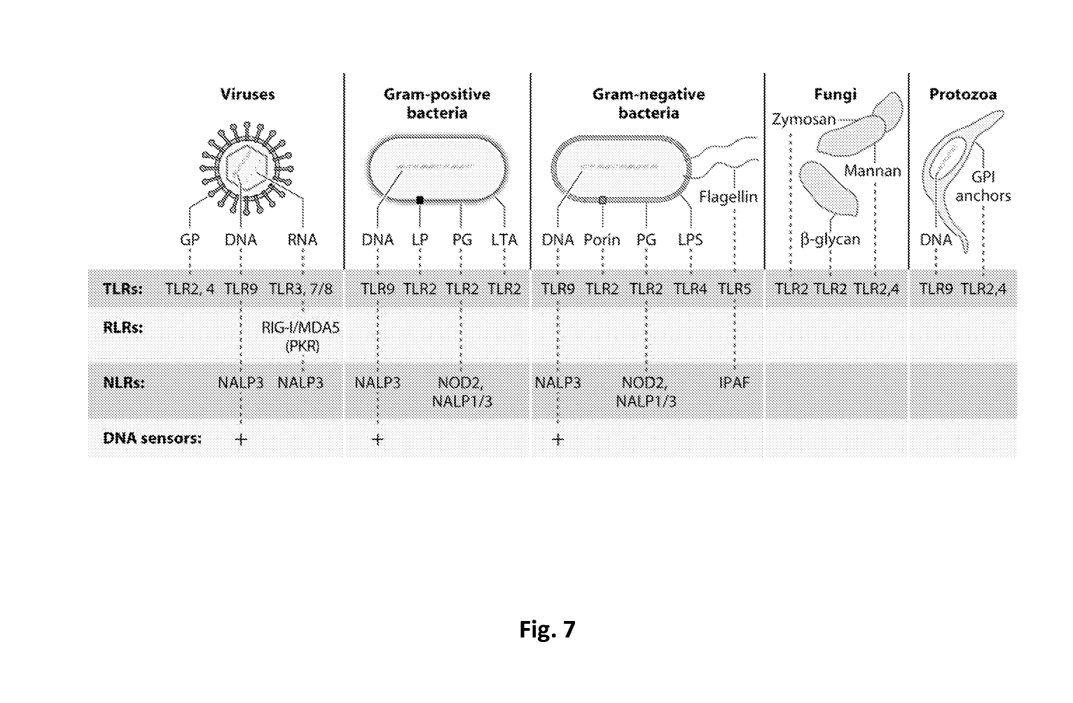

FIG. 8. Defining codes having different PAMP agonist combinations.

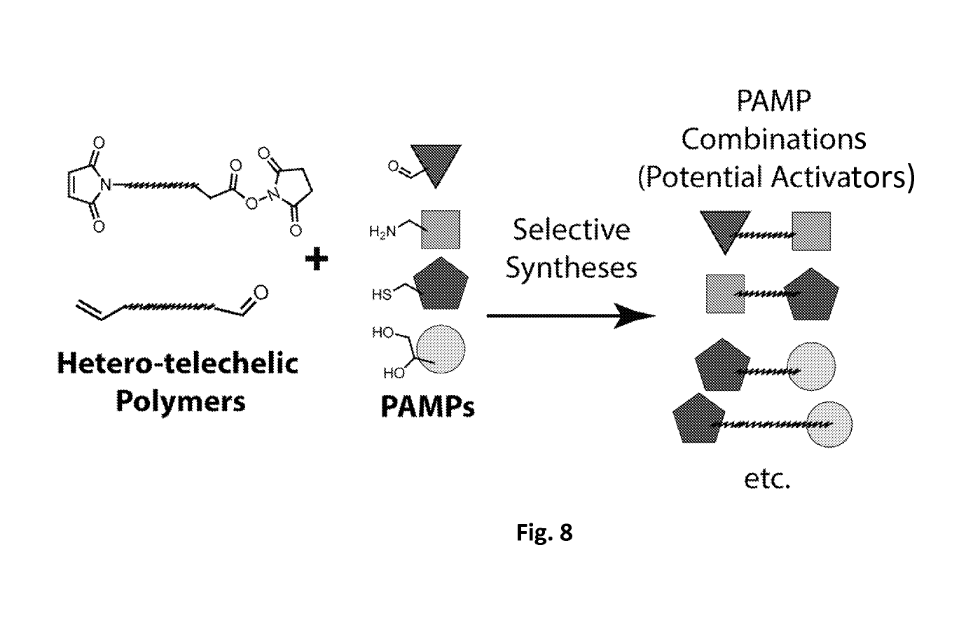

FIG. 9. Dimerization events occur in TLRs.

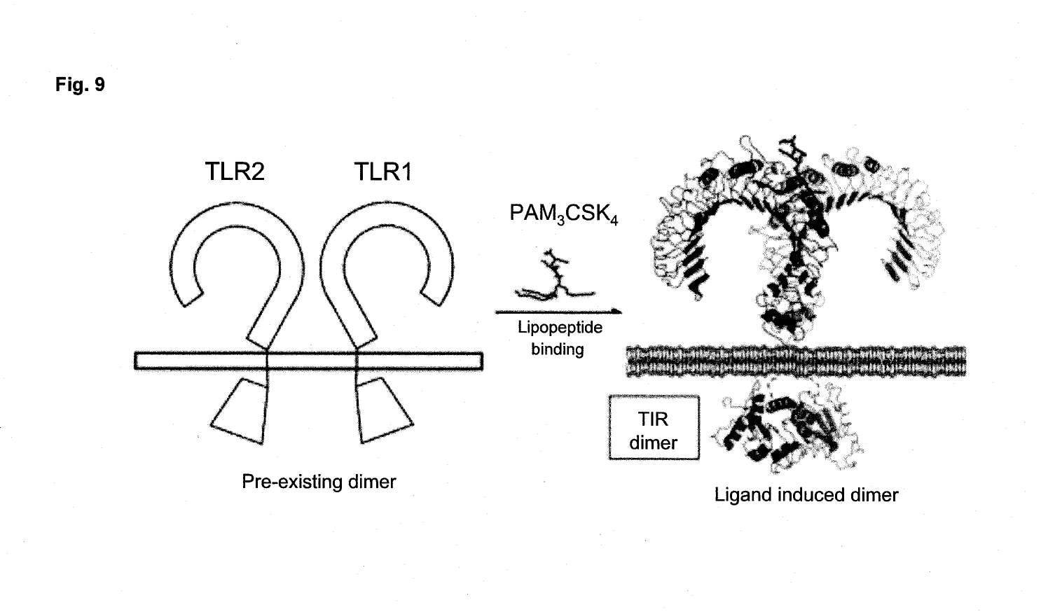

FIG. 10. Dimerization and molecular scale activation is quite common.

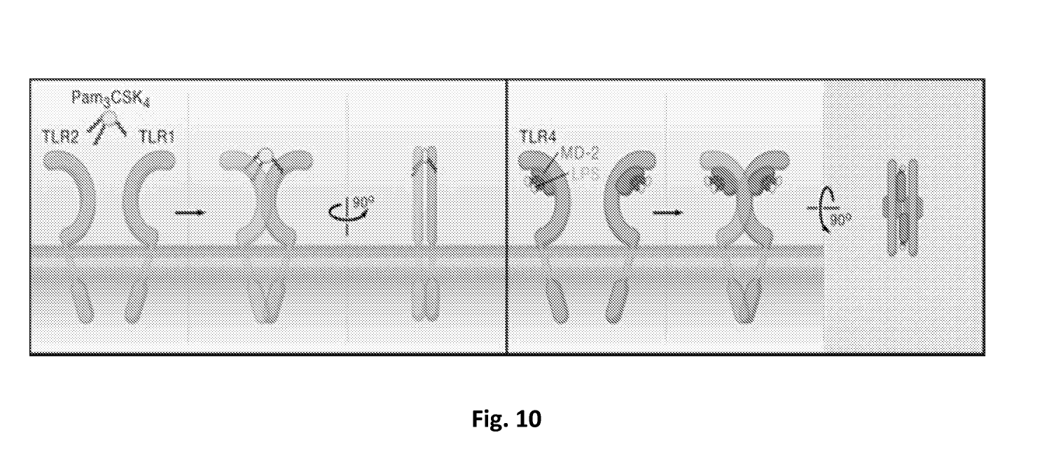

FIG. 11. TLR8 modeled as proceeding through a dimer.

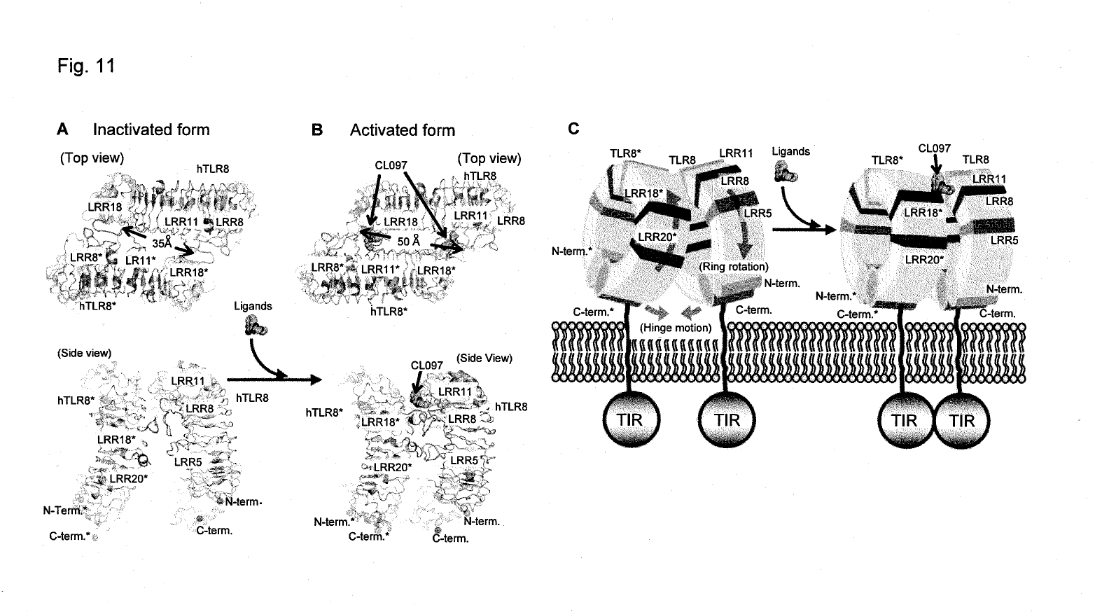

FIG. 12. Synergies are Partially Molecular.

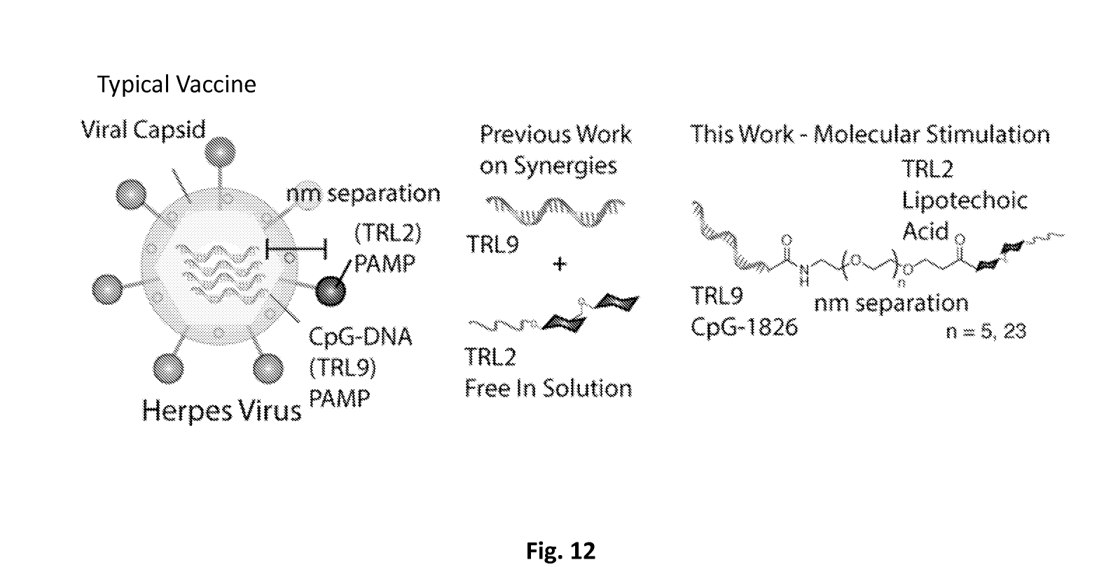

FIG. 13. A test structure to examine the model of molecular synergy.

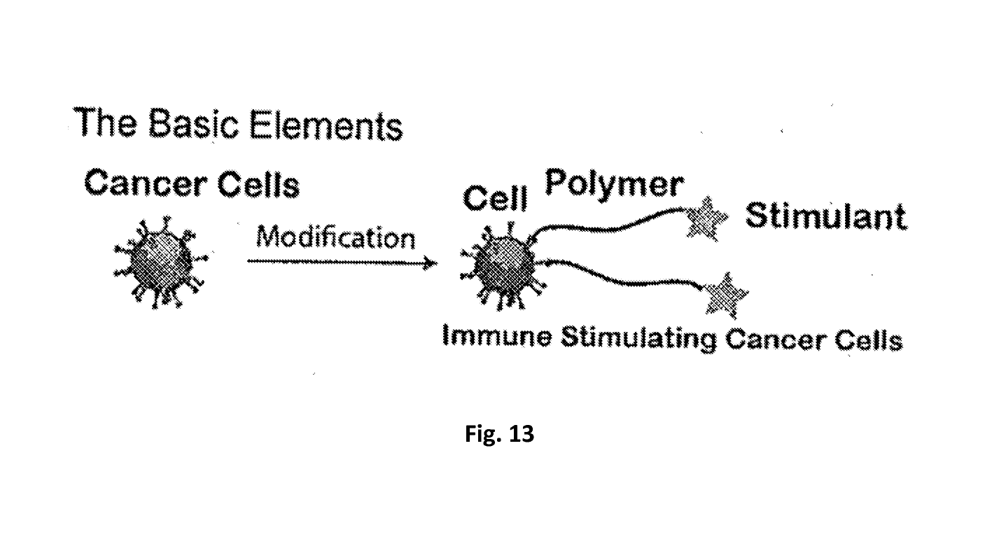

FIG. 14. CpG1826-Conjugate.

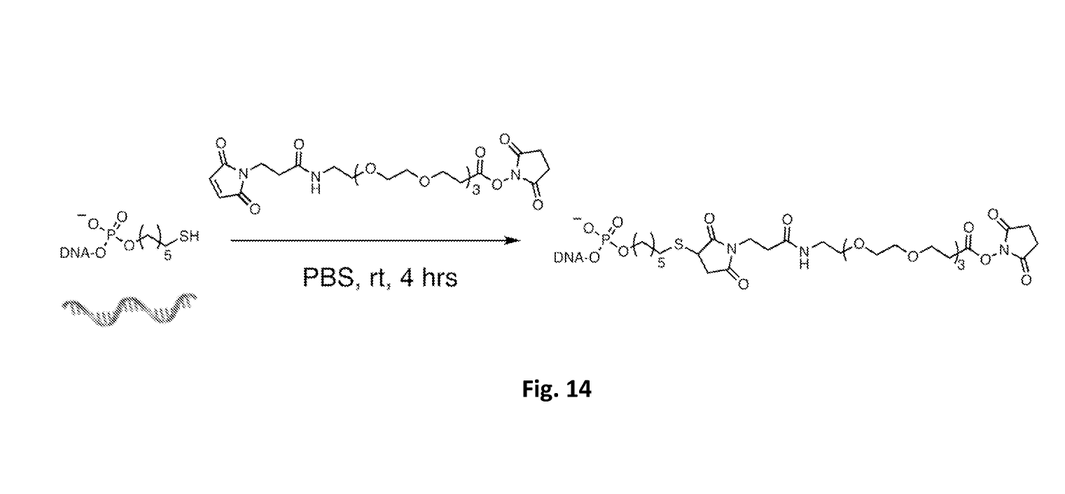

FIG. 15. TLR2 and TLR9 Co-localize. Synergistic interactions of TLR2/6 and TLR9 induce a high level of resistance to lung infection in mice (Duggan J. M. et al. 2011 J Immunol 186(10):5916-26).

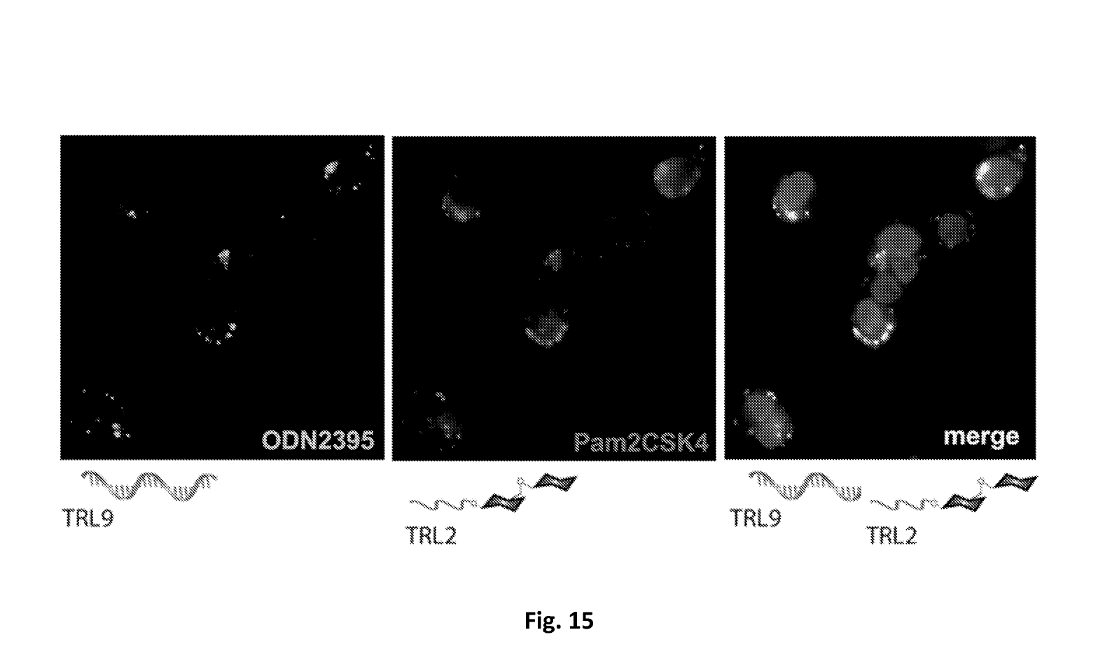

FIG. 16. Immune response stimulation is stronger when PAMPs are conjugated.

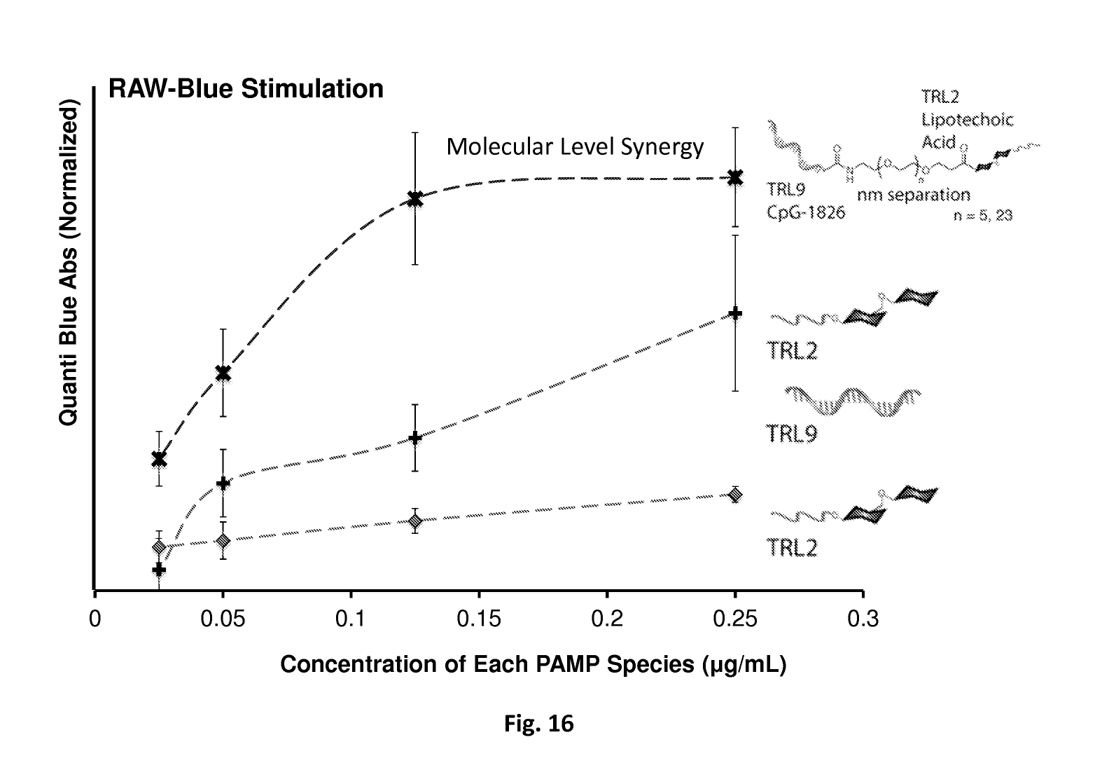

### DETAILED DESCRIPTION OF THE PREFERRED EMBODIMENT

The covalently linked, heterodimer molecules disclosed herein generate a larger immune response at a lower concentration than current immunostimulants. Additionally, the polarization of the stimulation is more diversified, a trait that is known to lead to improved amplification of the immune response and more durable anti-body titers.

Other combination immunostimulants directly and covalently attach two different PAMPs together or attach multiple PAMPs to macromolecular structures that are ill-defined or polydisperse with respect to extent of conjugation, molecular size, and inter-PAMP spacing. We control these aspects by using heterotelechelic polymers or linkers to attach two different PAMPs at discrete well-defined inter-PAMP distances.

The linear inter-PAMP spacing of the covalently linked, heterodimer molecules disclosed herein may vary. For example, in some embodiments, the spacing is from 10 to 500 Å, 10 to 400 Å, 10 to 300 Å, 10 to 200 Å, 10 to 100 Å, 20 to 100 Å, 20 to 50 Å, 30 to 100 Å or 30 to 50 Å.

PAMP molecules are well known in the art. (Kumar, H. et al. 2011 International Reviews of Immunol 30: 16-34). Rapid progress has been made not only in our understanding of the structure of TLRs but also in revealing the complexity of TLR-mediated signaling and in the identification of PAMPs derived from microbial pathogens such as mycobacteria, bacteria, viruses, fungi and parasites. Examples of PAMPs recognized by various TLRs are shown in Table I.

TABLE I
TLR Ligands and Cellular Location

| | Cellular | |
|---|---|---|
| TLR and (co-receptors) | location | Example TLR ligands |
| TLR1/2 | Cell surface | Triacyl lipopeptides |
| TLR2 (Dectin-1, | Cell surface | Peptidoglycan, lipoarabinomannan, |
| C-type lectin) | | hemagglutinin, phospholipomannan, |
| | | glycosylphosphophatidyl inositolmucin, |
| | | zymosan |
| TLR3 | Endosome | ssRNA virus, dsRNA virus, respiratory syncytial |
| | | virus, murine cytomegalovirus |
| TLR4 (MD2, CD14, | Cell surface | Lipopolysaccride, mannan, |
| LBP) | | glycoinositolphospholipids, envelope and fusion |
| | | proteins from mammary tumor virus and |
| | | respiratory syncytial virus, respectively, |
| | | endogenous oxidized phospholipids produced |
| | | after H5N1 avian influenza virus infection, |
| | | pneumolysin from streptococcus pneumonia, |
| | | paclitaxel. |
| TLR5 | Cell surface | Flagellin from flagellated bacteria |
| TLR6/2 (CD36) | Cell surface | Diacyl lipopeptides from mycoplasma), |
| | | lipoteichoic acid |
| TLR7 | Endolysosome | ssRNA viruses, purine analog compounds |
| | | (imidazoquinolines). RNA from bacteria from |
| | | group B streptococcus |
| TLR8 (human) | Endolysosome | ssRNA from RNA virus, purine analog |
| | | compounds (imidazoquinolines). |
| TLR9 | Endolysosome | dsDNA viruses herpes simplex virus and murine |
| | | cytomegalovirus, CpG motifs from bacteria and |
| | | viruses, hemozoin malaria parasite |
| TLR11 (mouse) | Cell surface | Uropathogenic bacteria, profillin-like molecule |
| | | from Toxoplasma gondii |

Several bacterial, viral, fungal and parasitic PAMPs sensed by TLRs are described below.

### Bacterial PAMPs Sensed by TLRs

Of the TLRs, TLR1, 2, 4, 5, 6, 7, and 9 are primarily dedicated to the recognition of various bacterial components. LPS is a major cell wall component of gram-negative bacteria and is primarily sensed by TLR4 complexed with another molecule known as MD2. Another essential major component of gram-positive bacteria is peptidoglycan, which is sensed by TLR2. Mycobacteria, another class of bacteria rich in lipoarabinomannan (LAM), are also sensed by TLR2. TLR2 (in conjugation with TLR1 or TLR6) senses diacyl or triacyl lipopeptides on bacteria, mycobacteria and mycoplasma. TLR5 and TLR9 sense the flagellin protein expressed by flagellated bacteria and bacterial/viral genomic DNA rich in unmethylated CpG, respectively. Group B streptococci, which reside in the phagosome, are recognized by TLR7. Also, bacterial RNA produced in the lysosomal compartment is likely to act as a PAMP for TLR7. Recognition of PAMPs by TLR1, TLR2, TLR4, TLR5 and TLR6 primarily induces the production of inflammatory cytokines, whereas TLR7 and TLR9 induce type I interferons.

### Viral PAMPs Sensed by TLRs

Nucleic acids (single stranded (ss)/double stranded (ds) RNA or ss/dsDNA) derived from viruses are recognized by several TLRs. DNA from herpes simplex virus (HSV), murine cytomegalovirus (MCMV), as well as CpG motifs containing synthetic oligonucleotides that contain unmethylated CpG DNA, are sensed by TLR9, which induces the production of type I interferons, most likely through plasmacytoid dendritic cells (pDCs). RNA from RNA viruses is sensed by TLR7 and TLR8 (the function of murine TLR8 is not known). In addition, several synthetic antiviral compounds, such as R848, imiquimod and loxoribine, are also sensed by TLR7 and TLR8. Furthermore, the synthetic analog of dsRNA, known as poly IC, is sensed by TLR3 which also activate adaptive immunity when used as an vaccine adjuvant. Another PAMP derived from viruses is a coat protein. The coat proteins of respiratory syncytial virus (RSV) and mouse mammary tumor virus (MMTV) are sensed by TLR4. However, in vivo studies show that TLR2 and TLR6 play an essential role in controlling RSV infection. The coat proteins of other viruses, such as the Measles virus hemagglutinin protein, are also sensed by TLR2, as is Vaccinia virus. Notably, this recognition induces the production of type I interferons by inflammatory monocytes.

### Fungal PAMPs Sensed by TLRs

Several fungi, such as Candida albicans and Aspergillus fumigatus, are sensed by several TLRs and induce inflammatory responses. However, this recognition requires additional receptors such as dectins, CD14, mannose receptors, and DC-SIGN. β-glucans are the primary component of the majority of fungal cell walls, including those of baker's yeast and some pathogenic fungi such as Candida albicans. These β-glucans are recognized by TLR2 in association with dectin-1. Glucuronoxylomannans, another fungal component sensed by CD14 and TLR4, also induce inflammatory responses.

### Protozoal PAMPs Sensed by TLRs

Protozoal infections are a serious problem in developing countries and cause diseases such as Toxoplasmosis (Toxoplasma gondii), malaria (Plasmodium species), leishmaniasis (Leishmania species), and sleeping sickness (Trypanosoma brucei). Unsaturated alkylacylglycerol and lipophosphoglycan (LPG) from Trypanosoma species and Leishmania species, respectively, are recognized by TLR2, and glycoinositolphospholipids and glycosylphosphatidylinositol anchors from Trypanosoma species, P. falciparum and T. gondii are recognized by both TLR2 and TLR4. The profilin-like protein of T. gondii is sensed by murine TLR11. The genomic DNA and hematin crystals of Trypanosoma and Plasmodium species, respectively, are sensed by TLR9.

### PAMP Mimetics

The term “PAMP mimetic” refers to a molecule that, although it does not occur in microorganisms, is analogous to a PAMP in that it can bind to a Pattern Recognition Receptors (PRR) and such binding can trigger an innate immune response. A PAMP mimetic can be a naturally-occurring molecule or a partially or totally synthetic molecule. As an example, and not by way of limitation, certain plant alkaloids, such as taxol, are PRR ligands, have an immunostimulatory effect on the innate immune system, and thus behave as PAMP mimetics. (Kawasaki et al. 2000 J Biol Chem 275(4): 2251-2254).

### Chemistry for Covalent Linkage of PAMPs

The present invention includes “conjugates” which comprise two or more PAMP molecules that are covalently linked, or noncovalently linked but in association with each other. Thus, hetermeric molecules of the present invention include, but are not limited to the following: protein/nucleic acid conjugates, nucleic acid/protein conjugates, nucleic acid/nucleic acid conjugates, peptide-mimetic/nucleic acid conjugates, nucleic acid/peptide mimetic conjugates, peptide mimetic/peptide mimetic conjugates, lipopolysaccharide/protein conjugates, lipoprotein protein conjugates, RNA protein conjugates, CpG-DNA/protein conjugates, nucleic acid analog/protein conjugates, and mannan/protein conjugates. To the extent that PAMPs identified in the future are comprised of yet other chemical classes, conjugates containing such chemicals can also be contemplated.

Methods for the conjugation of polypeptides, carbohydrates, lipids and DNA are well known. See e.g., U.S. Pat. Nos. 4,191,668; 5,162,515; 5,700,922; 5,786,461; 6,060,056; and Borel, Y. and Borel, H. 1988 J Clin Invest 82: 1901-1907. Non-protein PAMPs such as CpG or CpG-DNA, and lipopolysaccharides may be conjugated by conventional techniques. For example, PAMP conjugates may be linked through polymers such as PEG, poly-D-lysine, polyvinyl alcohol, polyvinylpyrollidone, immunoglobulins, and copolymers of D-lysine and D-glutamic acid. Conjugation of the PAMPs to the polymer linker may be achieved in any number of ways, typically involving one or more crosslinking agents and functional groups on the PAMPs.

Polypeptide PAMPs will contain amino acid side chains such as amino, carbonyl, or sulfhydryl groups that will serve as sites for linking the PAMPs to each other. Residues that have such functional groups may be added to the PAMPs. Such residues may be incorporated by solid phase synthesis techniques or recombinant techniques, both of which are well known in the peptide synthesis arts.

In the case of carbohydrate or lipid analogs, functional amino and sulfhydryl groups may be incorporated therein by conventional chemistry. For instance, primary amino groups may be incorporated by reaction with ethylenediamine in the presence of sodium cyanoborohydride and sulfhydryls may be introduced by reaction of cysteamine dihydrochloride followed by reduction with a standard disulfide reducing agent. In a similar fashion the polymer linker may also be derivatized to contain functional groups if it does not already possess appropriate functional groups.

Heterobifunctional crosslinkers, such as sulfosuccimmidyl(4-iodoacetyl)aminobenzoate, which link the .epsilon. amino group on the D-lysine residues of copolymers of D-lysine and D-glutamate to a sulfhydryl side chain from an amino terminal cysteine residue on the peptide to be coupled, are also useful to increase the ratio PAMPs in the conjugate.

### Linkers for Defined Linear Inter-PAMP Spacing

The term “linker” refers to any chemical entity that links one chemical moiety to another chemical moiety. Thus, something that chemically or physically connects different PAMP molecules is a linker. Examples of linkers include, but are not limited to, complex or simple hydrocarbons, nucleosides, nucleotides, nucleotide phosphates, oligonucleotides, polynucleotides, nucleic acids, amino acids, small peptides, polypeptides, carbohydrates (e.g., monosaccharides, disaccharides, trisaccharides), and lipids. A linker functions as a spacer.

### Example 1

### Inter-Immunostimulant Proximity Effects in the Synergistic Stimulation of Dendritic Cells

We report the discovery of proximity based synergies at discrete molecular distances between two signals in the activation of dendritic cells. Vaccines, the most successful immune therapies, operate through synergistic stimulation of dendritic cell receptors using spatio-controlled presentation of pathogen associated molecular patterns (PAMPs) in the form of attenuated whole viruses. The response pathway of synergistic PAMP combinations result in increased stimulation, however, linking agonists of these synergistic pathways as PAMP-heterodimers with discrete, linear inter-PAMP spacing has never been tested. Here we show the synthesis of heterodimers containing two dendritic cell agonists, lipotechoic acid and CpG-DNA-ODN 1826 covalently attached as PAMP-heterodimers with varied spacing from 32.5 to 95.6 Angstroms. The immunostimulatory effects of the PAMP-heterodimers have been tested in murine RAW-Blue and bone marrow derived dendritic cell lines. An increase in stimulation was observed for PAMP-heterodimers relative to an equimolar mixture of unconjugated P AMPs or each PAMP alone, and this increase was found to be dependent on the inter-PAMP proximity. This is the first observation of a molecular level proximity based synergy between these PAMP receptors produced by covalently attaching the PAMPs through a polymeric linker.

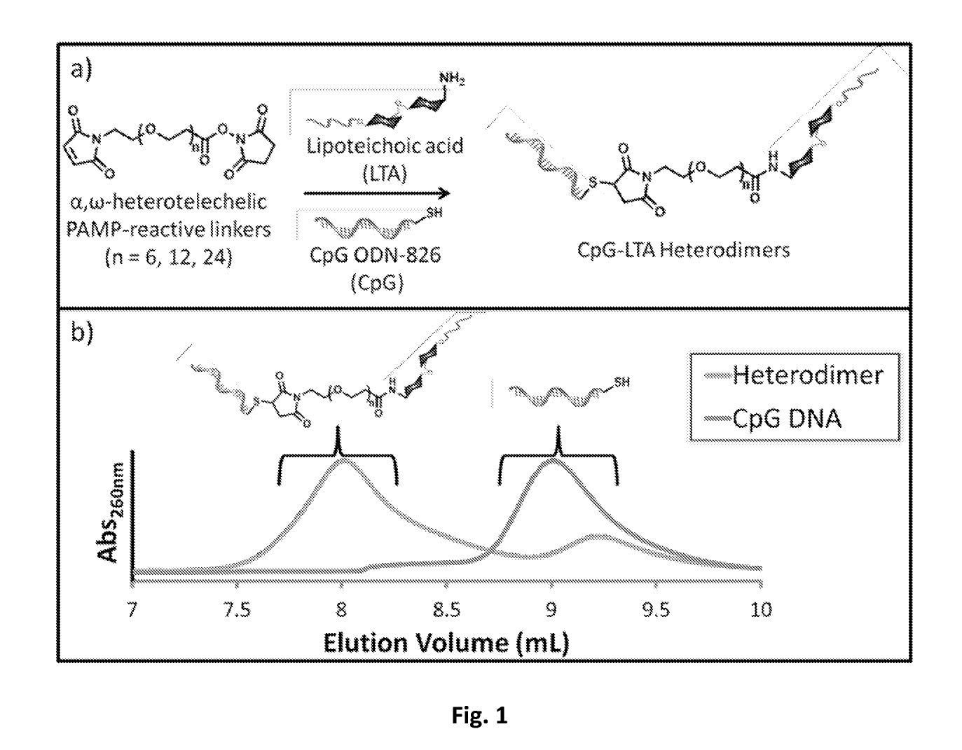

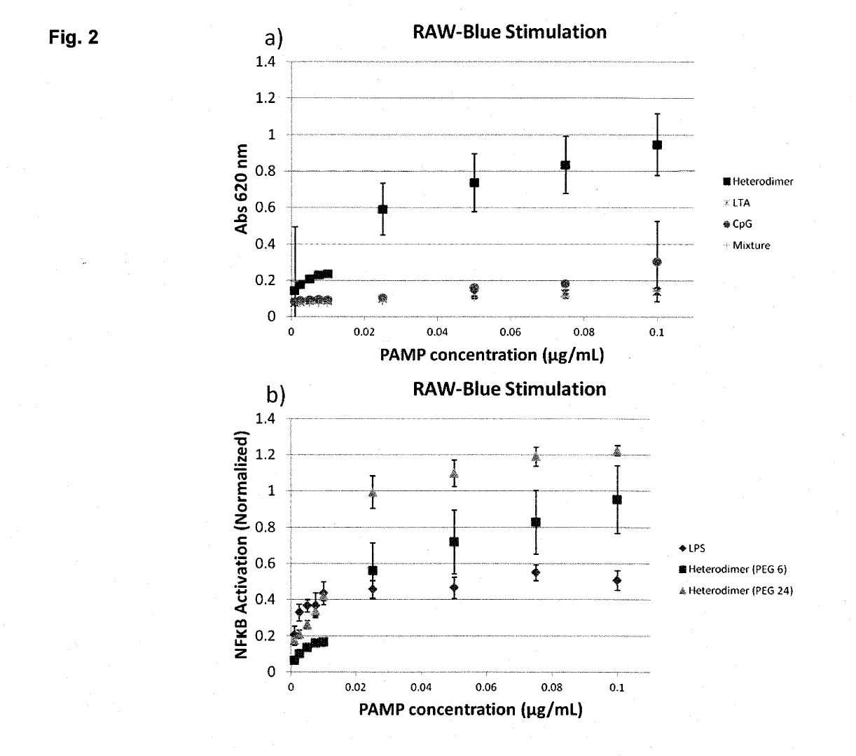

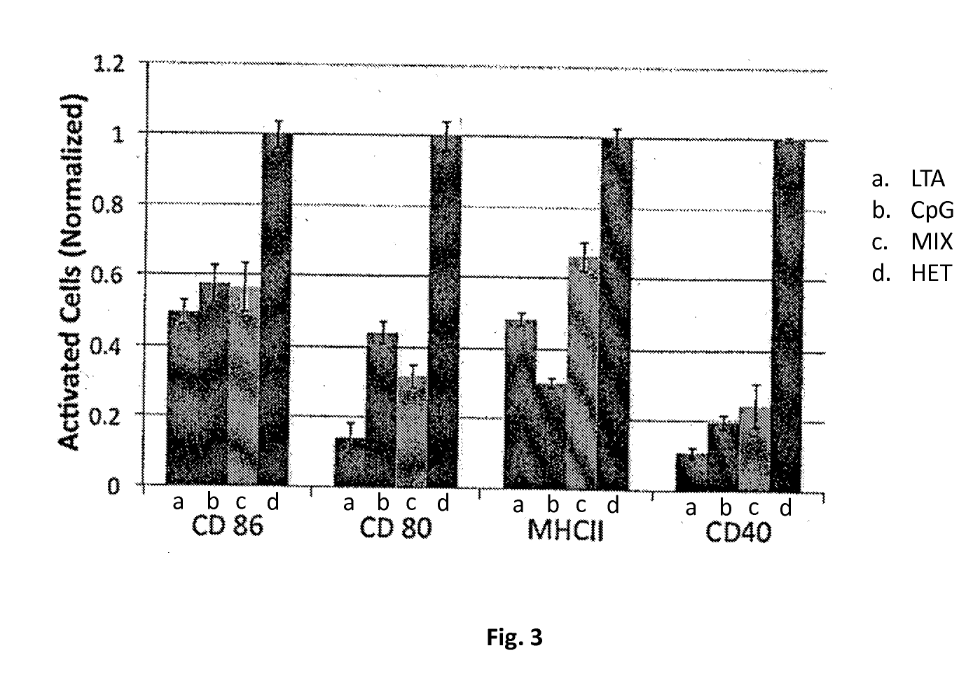

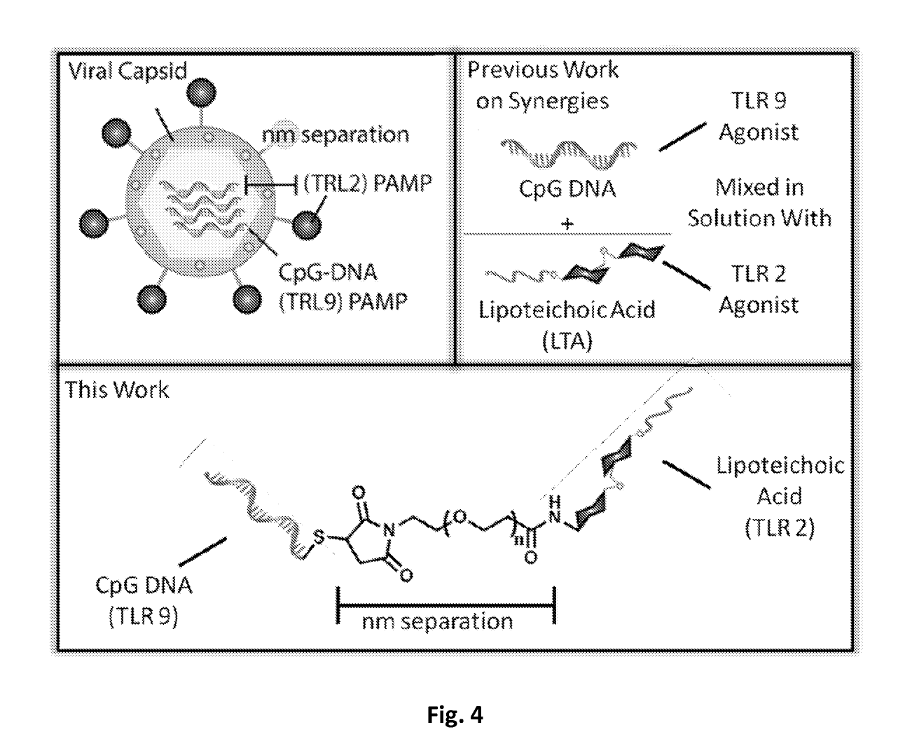

Here we show the synthesis of single molecule, polymer-linked, heterodimers of two dendritic cell agonists (FIG. 1). We have covalently linked agonists of two different Toll-Like Receptors (TLRs), 4 lipoteichoic acid (LTA), and CpG DNA-ODN 1826 (CpG), to form PAMP-heterodimers with varied inter-PAMP spacings (FIG. 4). The immunostimulatory effects of the PAMP-heterodimers have been tested in a murine RAW-Blue cell line and cellular targeting has been observed to occur through interaction with TLR 2 (FIG. 2). The PAMP-heterodimers were also tested in a Primary Bone Marrow Derived Dendritic Cell (BMDC) line, and an increase in CD86 (a protein involved in T-cell mediated immune amplification) was found with retention of other cell surface proteins critical to the immune response (FIG. 3).

We present a lipoteichoic acid (TLR 2 and 6 agonist) covalently attached to a CpG DNA fragment (TLR 9 agonist) using α,ω-heterotelechelic PEG based linkers (FIG. 1). The CpG DNA used was a Th1 polarizing endosomal TLR 9 agonist, CpG ODN 1826 (CpG) (Chu, R. S. et al. 1997 J Exp Med 186: 1623-1631). This was attached to the cell-surface TLR 2 and 6 agonist, lipoteichoic acid (LTA) which induces both Th1 and Th2 polarization (Akira, S, and Hemmi, H. 2003 Immunol Lett 85: 85-95). Lipoteichoic acid (LTA) is a class of glycosylated lipopolypeptide (Ginsburg, I. 2002 The Lancet Infectious Diseases 2: 171-179; Fischer, W. et al. 1990 Biochemistry and Cell Biology 68: 33-43). LTA is formed from a phosphate linked polymeric backbone comprising both polysaccharides and d-alanine residues. This macrostructure is linked, via ester bonds, to alkyl chains required for agonistic activity. As such, we sought to covalently link LTA at the alanine side-chain residues using N-hydroxy succinimidyl (NHS) activated esters. The CpG sequence was modified to contain a thiol at the 3′ end and FAM tag installed at the 5′ ends respectively. We envisioned that we could react the thiol end of the CpG with maleimide functionality on the LTA-PEG conjugates to complete the PAMP-heterodimer (FIG. 1).

The mammalian immune response is directed, in part, by cytokines secreted by antigen presenting cells (dendritic cells). A balance of cytokines results in the balance of antibody producing and effector cells found in the most potent vaccination strategies. Generally, activation of anyone TLR on a DC leads to stimulation and direction of the immune system. Simultaneous activation of multiple TLRs can result in additive or synergistic stimulatory effects and polarization of the immune response pathway can be controlled via this method (Napolitani, G. et al. 2005 Nat Immunol 6:769-776; Raman, V. S. et al. 2010 J Immunol 185: 1701-1710; Chen, K. et al. 2009 J Neuroimmunol 213: 69-77). This implies that stimulation of DCs with different combinations of PAMPs can control immune amplification in the DC mediated activation of naive T cells. As such, the immune system can be primed to illicit a humoral or cellular immune response with different applications requiring varied control over different combinations of pathways. Multiple pathways of activation are known for the general naive T cell type CD4+ (Th0, Th1, Th2) (Kim, C. H. et al. 2001 J Clin Invest 108: 1331-1339; Kapsenberg, M. L. 2003 Nat Rev Immunol 3:984-993) and polarization is possible (Ma, J. et al. 2007 J Immunol 178:7814-7821) but not required (Kovacs, B. et al. 2002 Proc Natl Acad Sci USA 99: 15006-15011) for activation of the CD8+ class as well. Understanding each of these pathways is important, as different applications require controlling the activation or suppression of different combinations of pathways. For example, a Th1 and CD8+ response with suppression of Th2 polarization has traditionally been targeted in cancer immunotherapy (Schuler-Thurner, B. et al. 2002 J Exp Med 195: 1279-1288), Th1 and/or Th2 activation is generally desired in vaccine formulation (Gupta, R. and Siber, G. 1995 Vaccine 13:1263-1276) and suppression of Th2 is useful in asthma treatments (Shinohara, M. L. and Cantor, H. 2007 Nature Medicine 13: 536-538). To date several approaches have been developed that combine multiple PAMP signals in a single immunostimulant including virus, nanoparticle (Reddy, S. T. et al. 2007 Nat Biotechnol 25: 1159-1164), and dendrimer (Shukla, N. M. et al. 2012 PLoS ONE 7: e43612) motifs. Additionally, well-defined and relatively short homodimer spacings have been achieved resulting in modulation of immunostimulation through TLRs 7 and 8 (Shukla, N. M. et al. 2012 J Med Chem 55: 1106-1116). Therefore, we sought to explore the effect of inter-PAMP proximity on TLR activation. We used α,ω-heterotelechelic PEG linkers to form PAMP-heterodimers consisting of immunostimulants from two different pathways (Th2 and Th1) with corresponding TLRs in two different cellular locations, cell-surface and endosomal compartments.

The PAMP-heterodimers were tested across two different cell lines, RAW-Blue cells and murine Bone Marrow Derived Dendritic Cells (BMDCs). It was found that the PAMP-heterodimers activated each cell line to a greater extent than a solution containing unconjugated PAMPs mixed together at equivalent concentrations. In addition the PAMP-heterodimers exhibited a 10-fold increase in immunostimulatory activity relative to lipopolysachamide A (LPS), one of the most potent known immunostimulants.

The PAMP-heterodimers provided greater up regulation of cell surface markers and release of cytokines associated with activation of the immune response in bone marrow derived dendritic cells (BMDCs). Our working hypothesis is that there is a molecular level synergy between TLR2 and TLR9. This synergy occurs in the herpes virus, which successively activates TLR2 and then TLR9.

### Synthesis of LTA-PEG Conjugates

To synthesize the PAMP-heterodimers we used PEG polymers of varied lengths with NHS and maleimide end-groups (PEG6 and PEG24 for PEG polymers with 6 and 24 repeat units respectively). The construction of the PAMP-heterodimers required several unique conditions. Strict control of the solution pH was required during all stages of the bioconjugation processes as the LTA is prone to dealkylation or dealanation under acidic or basic pH. LTA from Bacillus subtilis was conjugated to PEG6 or PEG24 via coupling of the alanine side-chain to the NHS ester end-group. In a typical reaction PEG6 (0.1 mg, 100 mg/mL, DMSO) was added to LTA (2 mg, 10 mg/mL, PBS, pH 7.4, 1 mM EDTA) and the reaction was incubated for 1 h at room temperature. The LTA-PEG6 conjugate was subjected to centriprep purification (3 kDa MWCO, DPBS, pH 7.4, 1 mM EDTA) to remove the excess PEG6. LTA-PEG24 was synthesized via an identical method and we characterized the conjugates by UV/Vis spectroscopy and SDSPAGE. The LTA-PEG6 and LTAPEG24 conjugates were found to have an Absₘₐₓ at 256 nm, and the concentration of the conjugate was quantified by this method for use in the next step.

The LTA-PEG6 and LTA-PEG24 conjugates were further elaborated to produce LTA-PEG6-CpG and LTA-PEG24-CpG heterodimers. In a typical reaction, purified LTA-PEG6 conjugate (2 mg, 10 mg/mL, DPBS, pH 7.4, 1 mM EDTA) was added to CpG (100 μg, 1 mg/mL, DPBS, pH 7.4, 1 mM EDTA), and the reaction mixture was incubated for 24 h at 30° C. with constant shaking. The crude LTA-PEG6-CpG heterodimer was purified via fast protein liquid chromatography (Superdex G75, DPBS, 0.2 mL/min), and the purified PAMP-heterodimer was analyzed by SDS-PAGE and quantified by UV/Vis. A similar procedure was used to synthesize the LTA-PEG24-CpG heterodimer. For UV/Vis characterization an Absₘₐₓ was found at 260 and 495 nm. The absorbance at 260 nm primarily corresponded to the CpG fragment (with a 10-fold smaller contribution from the LTA and PEG) and the absorbance at 495 corresponded to the FAM tag on the CpG DNA. As such, the absorbance at 495 nm was used to quantify the conjugate for comparison to known standards quantified via the same method for CpG or by mass for LTA.

The immunostimulatory activity of the PAMP-heterodimers was investigated using RAW-Blue cells and bone-marrow derived dendritic cells (BMDCs). The RAW-Blue assay (Invivogen, CA) is the simplest measure of NFκB activation, a broad method for determining immune cell stimulation. RAW-Blue cells are a murine macrophage derived cell line that express TLRs 1-9 with the exception of TLR 5. Secreted alkaline phosphatase (SEAP) is produced in response to activation of the NFκB pathway and may be quantified in the cell media using the Quanti Blue detection reagent. The effect of covalent attachment in the P AMP-heterodimer was explored. The RAW-Blue cell line was stimulated with each PAMP including the PAMP-heterodimers over concentrations ranging from 1 to 100 ng/mL (with respect to CpG in the case of the PAMP-heterodimers and PAMP mixture). Cells were seeded on a 96 well plate at a density of 100,000 cells/well in 200 μl of DMEM high glucose with 10% heat inactivated FBS, and 2 mM glutamine, and the cultures were allowed to incubate for 24 h. Next, the original media was removed and replaced with 190 μL of media and 10 μL of a 20× solution of each sample in DPBS, pH 7.4. After 15 h, the cell cultures were assayed for SEAP by incubating a 10 μL aliquot of each sample of culture media with 200 μL of the QUANTI-BLUE™ SEAP detection reagent for 2.5 h before colorimetric assay at 620 nm.

The PAMP-heterodimers stimulated the cells to a greater extent than mixtures of each PAMP combination or each PAMP alone (FIG. 2). This could be due to multiple mechanisms that increase activation: (1) The alkyl chains of the LTA could be providing cellular targeting for the CpG fragment by binding TLRs 2 and/or 6 or by intercalating the cell membrane; (2) The CpG DNA could be providing this same targeting through binding to CpG cell surface receptors such as DEC-205; and/or (3) There could be a prearranged order of the CpG and LTA cell surface receptors such that binding of one PAMP enhances binding of a second PAMP in close proximity. In addition, the LTA-PEG24-CpG heterodimer exhibited greater stimulation than the LTA-PEG6-CpG heterodimer. This result is intriguing because it could imply that after binding one TLR the longer heterodimer can access more of the cell surface to bind to a second TLR thereby completing the synergistic pair.

To better understand the effect our PAMP-heterodimers on active APCs, we tested the LTA-PEG6-CpG heterodimer against Bone Marrow Derived Dendritic Cells (BMDCs). BMDCs were derived from monocytes isolated from mice as described previously, and cultured in complete media (RPMI, 10% heat inactivated FBS, 30 ng/mL GMCSF) for 3 days. On the 3rd day the volume of the cell culture was doubled by addition of complete media and the cells were cultured an additional 3 days before use. The BMDCs were seeded on 24-well plates at a density of 600,000 cells/well. Each PAMP, including the LTA-PEG6-CpG heterodimer, was incubated with BMDCs at a concentration of 100 ng/mL for 18 h. A mixture of LTA and CpG at a concentration of 100 ng/mL each was also tested. Using immunohistochemical staining, we observed an increase in cell surface proteins corresponding to activation for the LTA-PEG6-CpG heterodimer relative to mixtures of unconjugated PAMPs (FIG. 3). Namely, we observed an increase in all T-cell adhesion proteins and MHCII for the LTA-PEG6-CpG heterodimer relative to all other PAMPs or unconjugated PAMP mixtures tested. The cell surface protein MHCII is involved with antigen presentation and is present to a significant extent in the unstimulated case. MHCII is upregulated when the cells are stimulated with the LTA-PEG6-CpG heterodimer. The stimulation profile we observed is indicative of an increase in antigen cross-presentation and T-cell expansion based on the increase in T cell adhesion proteins and MHCII. We therefore expect that the PAMP-heterodimers will perform as superior immunostimulants relative to either PAMP alone or unconjugated mixtures thereof.

### Conclusions

In summary, we have synthesized two PAMP-heterodimers containing CpG and LTA linked at two different inter-PAMP proximities. For the LTA-PEG-CpG heterodimers we have observed greater increases in immune stimulation proceeding through molecular synergy of a hypothesized dimerization. There is synergistic stimulation of the NFκB pathway in RAW-Blue cells, which might occur via cellular targeting through TLRs 2 and 6 or DEC-205. In BMDCs, increases in proteins involved with T-cell expansion were observed. Controlling PAMPs at a molecular level via hetero-dimerization shows that much can still be learned about stimulating immune cells can be optimized. The response seen in our PAMP heterodimers indicates that controlling the spatio-temporal presentation of PAMPs to TLRs and other Pattern Recognition Receptors (PRRs) can dramatically alter the stimulation profile of immune cells.

### Example 2

### Synthesis of IMQ-PEG6-Cpg

Imiquimod (IMQ, 100 μL, 10 mg/mL DMSO) was added to the PEG6 linker (1 mg, 100 mg/mL, DMSO) and the reaction was incubated at 40° C. with constant shaking to obtain the crude reaction product (IMQ-PEG6) after 3 h. Next, CpG (100 μg, 1 mg/mL, DPBS, pH 7.4) was added and the reaction was incubated for an additional 24 h before purification by FPLC.

### Example 3

### Synthesis of IMQ-PEG6-LTA

LTA (2 mg, 10 mg/mL) was incubated with succinimidyl acetyl thiopropionate (2 μL, 16 mg/mL, DMF) in PBS pH 7.4 with 1 mM EDTA at room temperature for 3 h. The resulting mixture was purified by dialysis (3 kDa MWCO) against PBS pH 7.4 with 1 mM EDTA before dilution in 0.5 mL of 0.5M hydroxylamine with 25 mM EDTA. The solution was incubated for 1 h and purified by dialysis (3 kDa MWCO) against PBS pH 7.4 with 1 mM EDTA to obtain purified thiolated LTA (LTA-SH). Thiols on LTA-SH were quantified via Ellman's Assay (0.6 thiols/LTA) and quantified by UV/Vis (Absmax=256 nm). LTA-SH (1 mg, 1 mg/mL, DPBS, pH 7.4) was incubated with a solution of IMQ-PEG6 (1 mg each IMQ and PEG6, 10 mg/mL, DMSO) synthesized as above for 24 h at room temperature. The resulting conjugate was purified by FPLC and characterized by UV/Vis and GPC.

### Example 3

### Synthesis of MPLA-PEG6-CPG

Monophosphoral Lipid A (MPLA, 1 mg) was added to a solution of the PEG6 linker (0.1 mg, 1 mg/mL, DMSO) and the reaction was incubated at 40° C. for 3 h with constant shaking. The resulting MPLA-PEG6 conjugate was purified by dialysis (3 kDa MWCO) against DPBS pH 7.4 with 1 mM EDTA. CPG (100 μg, 1 mg/mL, DPBS, pH 7.4) was incubated with the purified MPLA-PEG6 conjugate at 40° C. for 24 h. The crude reaction mixture was purified by FPLC and the resulting MPLA-PEG6-CPG conjugate was characterized by UV/Vis and GPC.

### Example 4

### Other Combinations of Heteromeric Molecules of Covalently Linked PAMP Molecules

Other combinations include heterodimers with: Flagellin or flagellin peptide (flg2); PAM2CSK4; PAM3CSK4; poly (I:C) RNA or other double stranded RNA sequences; types of CPG, for example ODN 2006; Imidazoquinolines, such as resiquimod; lipopolysaccharides and bacterial polysaccharides.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described herein. In addition, the materials, methods, and examples described herein are illustrative only and are not intended to be limiting.

What is claimed is:

1. A molecule comprising two or more different pathogen associated molecular patterns (PAMPs), wherein the two or more PAMPs are linked together with discrete, linear inter-PAMP spacing.

2. The molecule of claim 1, wherein the two or more different PAMPs are linked together with a heterotelechelic polymer.

3. The molecule of claim 1, wherein the linear inter-PAMP spacing is from 10 to 100 Å.

4. The molecule of claim 3, wherein the linear inter-PAMP spacing is from 32.5 to 95.6 Å.

5. The molecule of claim 1, wherein the two or more different PAMPs comprise lipoteichoic acid and a CpG-DNA fragment.

6. An immunogenic composition comprising the molecule of claim 1.

7. A method of stimulating an immune response in vivo in a subject comprising administering the immunogenic composition of claim 6 to said subject.

8. The method of claim 7, wherein said subject has a tumor or a cancer.

9. A method of stimulating an immune response ex vivo comprising administering the immunogenic composition of claim 6 to cells.

10. A method of synthesizing an immunostimulant comprising:
(a) identifying 2 or more different PAMPS,
(b) covalently linking the 2 or more different PAMPS together,
(c) varying spacing between the 2 or more different PAMPs, and
(d) testing the effect of spacing between the 2 or more different PAMPS to identify an optimal spacing that results in immune stimulation.
